(19) United States
(12) Patent Application Publication
(10) Pub. No.: US 2005/0170490 A1
(43) Pub. Date: Aug. 4, 2005

(54) Processes for manufacturing optical analysis discs with molded microfluidic structures and discs made according thereto

(76) Inventors: YihFar Chen, Kaohsiung City (TW); Chih-Hsin Shih, Aliso Viejo, CA (US); James Rodney Norton, Santa Ana, CA (US); Mark Oscar Worthington, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/353,777

(22) Filed: Jan. 27, 2003

Related U.S. Application Data
(60) Provisional application No. 60/353,726, filed on Jan. 31, 2002.

(51) Int. Cl. C12M 1/34; C12M 3/00; G01N 21/00; G01N 31/22

(52) U.S. Cl. 435/287.1; 422/58

(57) ABSTRACT

A process for manufacturing optical analysis discs with microfluidic structures and a disc made according thereto. The process includes the steps of providing a lens disc, patterning an intermediate adhesive layer with a pattern corresponding to predetermined microfluidic structures, providing a cover disc having molded microfluidic structures, and applying the cover disc onto the intermediate adhesive layer to match the molded microfluidic structures to the pattern to bond the cover disc onto the lens disc.

### CROSS-REFERENCE TO RELATED APPLICATIONS

This application claims the benefit of priority from U.S. Provisional Application Ser. No. 60/353,726 filed Jan. 31, 2002, which is herein incorporated by reference in its entirety.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND OF THE INVENTION

1. Field of the Invention

This invention relates in general to optical disc manufacturing and, in particular, to methods of making microfluidic structures. More specifically, but without restriction to the particular embodiments hereinafter described in accordance with the best mode of practice, this invention relates to processes for manufacturing optical analysis discs with microfluidic structures molded therein and to the discs made according to these methods.

2. Discussion of the Background Art

CDs and DVDs enable large amounts of data to be stored and quickly retrieved. Audio, visual, and computer program data are frequently stored on CDs or DVDs in a digital format. The end appearance of stored visual data is not readily apparent upon visual inspection. Thus, to extract visual information from a disc, a CD or DVD reader collects a stream of data that contains an encoding of the visual image. The functional aspects of a CD or DVD can be better understood with a review of CDs.

A CD is a fairly simple piece of plastic, about four one-hundredths ( 4/100) of an inch (1.2 mm) thick. Most of a CD consists of an injection-molded piece of clear polycarbonate plastic. During manufacturing, this plastic is impressed with microscopic bumps arranged as a single, continuous, extremely long spiral track of data. Once the clear piece of polycarbonate is formed, a thin, reflective aluminum layer is sputtered onto the disc, covering the bumps. Then a thin acrylic layer is sprayed over the aluminum to protect it. The label is then printed onto the acrylic.

A CD has a single spiral track of data, circling from the inside of the disc to the outside. The fact that the spiral track starts at the center means that the CD can be smaller than 4.8 inches (12 cm) if desired. The data tracks are approximately 0.5 microns wide, with 1.6 microns separating one track from the next, and the elongated bumps that make up the track are each 0.5 microns wide, a minimum of 0.83 microns long and 125 nanometers high. Frequently, data is described as encoded in pits on a CD instead of bumps. Usually, the marks appear as pits on the aluminum side, but on the side the laser reads from, they are bumps. However, the scheme can be reversed.

The CD player has the job of finding and reading the data stored on the CD. Considering how small the bumps are, the CD player is an exceptionally precise piece of equipment. The drive consists of three fundamental components. A drive motor spins the disc and is precisely controlled to rotate, typically, between 200 and 500 rpm depending on which track is being read. A laser and a lens system focus in on and read the bumps. A tracking mechanism moves the laser assembly so that the laser's beam can follow the spiral track. The tracking system has to be able to move the laser at micron resolutions.

Inside the CD player, there is frequently a degree of computer technology involved in forming the data into understandable data blocks and sending them either to the DAC (in the case of an audio CD) or to the computer (in the case of a CD-ROM drive). The fundamental job of the CD player is to focus the laser on the track of bumps. The laser beam passes through the polycarbonate layer, reflects off the aluminum layer and hits an opto-electronic device that detects changes in light. The bumps reflect light differently than the lands (the rest of the aluminum layer), and the opto-electronic sensor detects that change in reflectivity. The electronics in the drive interpret the changes in reflectivity in order to read the bits that make up the bytes.

One difficult task is keeping the laser beam centered on the data track. This centering is the job of the tracking system. The tracking system, as it plays the CD, has to continually move the laser outward. As the laser moves outward from the center of the disc, the bumps move past the laser faster. This happens because the linear, or tangential, speed of the bumps is equal to the radius times the speed at which the disc is revolving (rpm). Therefore, as the laser moves outward, the spindle motor must slow the speed of the CD. That way, the bumps travel past the laser at a constant speed, and the data comes off the disc at a constant rate.

Those endeavoring in the field of optical storage discs and drives have typically not heretofore addressed the technical issues relating to implementing micro-fluidic structures in optical storage discs for clinical diagnostic purposes, or for performing chemical, biochemical, or chemical assays.

Generally, microfluidic structures are made in flat articles by overlapping of cut or stamped intermediate layers between cover and substrate layers closing the channels and the chambers defined by cut or stamped portions of the intermediate layers thereby determining a microfluidic configuration.

### SUMMARY OF THE INVENTION

It is an object of the present invention to overcome limitations in the known art.

The present invention is directed to a process for manufacturing optical analysis discs with molded microfluidic structures and to optical analysis discs made according thereto.

More specifically, the present invention is directed to a process for manufacturing optical analysis discs with microfluidic structures. This process includes the steps of providing a lens disc; patterning an intermediate adhesive layer so as to have a pattern corresponding to predetermined microfluidic structures; providing a cover disc having molded microfluidic structures; and applying the cover disc onto the intermediate adhesive layer so as to match the molded microfluidic structures to the pattern to bond the cover disc onto the lens disc.

The present invention is also directed to a manufacturing process for optical analysis discs as defined above, wherein the intermediate adhesive layer is patterned according to capturing chemistry spots.

According to a further aspect of the invention, in the process for manufacturing the intermediate adhesive layer is applied by a transfer adhesive technique or by a printing technique and the intermediate adhesive layer is preferably made of a thermoglue.

According to a preferred embodiment, in the above defined process for manufacturing optical analysis discs, the step of providing a cover disc having molded microfluidic structures inside includes the steps of (1) providing a mastering support, (2) coating the mastering support with a photoresist composition, (3) transferring onto the mastering support a pattern design of microfluidic structures, (4) developing the photoresist composition according to the pattern design, (5) depositing a metal layer onto the mastering support until a plated stamper is obtained, preferably by a vacuum deposition technique, (6) peeling of the plated stamper, and (7) molding the microfluidic structures in a cover disc according the plated stamper.

Preferably, the mastering support is a mastering glass which is prepared by a process of cleaning.

In a preferred embodiment, the photoresist composition is selected in a group consisting of AZ5412, AZ4620, SU8-5, SU8-25, SU8-50, SU8-100. The coating of such photoresist composition is advantageously carried out by dipping, spray coating, or spin coating.

In order to possibly obtain a multi-layer microfluidic structure, the steps of coating the mastering support with a photoresist composition, transferring onto the mastering support a pattern design of microfluidic structures, and developing the photoresist composition according to the pattern design can be repeated.

The invention is further directed to an optical analysis disc made according the process above. The disc includes a lens disc, a patterned adhesive as intermediate layer for disc bonding, and a cover disc with molded microfluidic structures.

Preferably, in the optical analysis disc the lens disc comprises signaling tracks and the patterned adhesive is patterned also according to capturing chemistry spots. Further, the patterned adhesive layer is preferably a thermoglue.

This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 20, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 17, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6,2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; U.S. patent application Ser. No. 10/307,263 entitled “Magneto-Optical Bio-Discs and Systems Including Related Methods” filed Nov. 27, 2002; U.S. patent application Ser. No. 10/______ entitled “Method and Apparatus for Visualizing Data” filed Jan. 13, 2003; U.S. patent application Ser. No. 10/______ entitled “Methods and Apparatus for Extracting Data From an Optical Analysis Disc” filed on Jan. 14, 2003; U.S. patent application Ser. No. 10/______ entitled “Optical Discs Including Equi-Radial and/or Spiral Analysis Zones and Related Disc Drive Systems and Methods” filed on Jan. 15, 2003; and U.S. patent application Ser. No. 10/______ entitled “Bio-Safe Dispenser and Optical Analysis Disc Assembly” filed Jan. 17, 2003. All of these applications are herein incorporated by reference in their entireties. They thus provide background and related disclosure as support hereof as if fully repeated herein.

The above described methods and apparatus according to the present invention as disclosed herein can have one or more advantages which include, but are not limited to, simple and quick on-disc processing without the necessity of an experienced technician to run the test, small sample volumes, use of inexpensive materials, and use of known optical disc formats and drive manufacturing. These and other features and advantages will be better understood by reference to the following detailed description when taken in conjunction with the accompanying drawing figures and technical examples.

### BRIEF DESCRIPTION OF THE DRAWINGS

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1 is a pictorial representation of a bio-disc system;

FIG. 2 is an exploded perspective view of a reflective bio-disc;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17 is a pictorial graphical representation of the transformation of a sampled analog signal to a corresponding digital signal that is stored as a one-dimensional array;

FIG. 18 is a perspective view of an optical disc with an enlarged detailed view of an indicated section showing a captured white blood cell positioned relative to the tracks of the bio-disc yielding a signal-containing beam after interacting with an incident beam;

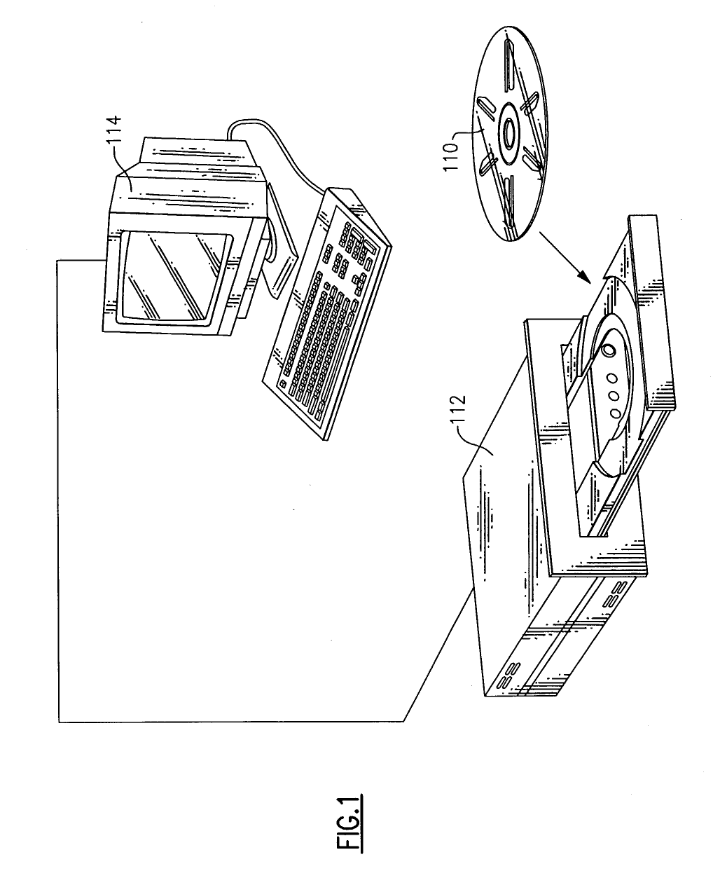

FIG. 1 9A is a graphical representation of a white blood cell positioned relative to the tracks of an optical bio-disc;

FIG. 19B is a series of signature traces derived from the white blood cell of FIG. 19A;

FIG. 20 is a graphical representation illustrating the relationship between FIGS. 20A, 20B, 20C, and 20D;

FIGS. 20A, 20B, 20C, and 20D, when taken together, form a pictorial graphical representation of transformation of the signature traces from FIG. 19B into digital signals that are stored as one-dimensional arrays and combined into a two-dimensional array for data input;

FIG. 21 is a logic flow chart depicting the principal steps for data evaluation according to processing methods and computational algorithms related to the present invention;

FIG. 22 is a an exploded perspective view of a reflective bio-disc made according the process for manufacturing of the present invention; and

FIG. 23 is a partial longitudinal cross sectional view of the optical bio-disc illustrated in FIG. 22 showing the microfluidic structures thereof.

### DETAILED DESCRIPTION OF THE INVENTION

The present invention is directed to disc drive systems, optical bio-discs, image processing techniques, counting methods and related software, x, y, and z. Each of these aspects of the present invention is discussed below in further detail.

Drive System and Related Discs

FIG. 1 is a perspective view of an optical bio-disc 110 according to the present invention as implemented to conduct the cell counts and differential cell counts disclosed herein. The present optical bio-disc 110 is shown in conjunction with an optical disc drive 112 and a display monitor 114. Further details relating to this type of disc drive and disc analysis system are disclosed in commonly assigned and co-pending U.S. patent application Ser. No. 10/008,156 entitled “Disc Drive System and Methods for Use with Bio-discs” filed Nov. 9, 2001 and U.S. patent application Ser. No. 10/043,688 entitled “Optical Disc Analysis System Including Related Methods For Biological and Medical Imaging” filed Jan. 10, 2002, both of which are herein incorporated by reference.

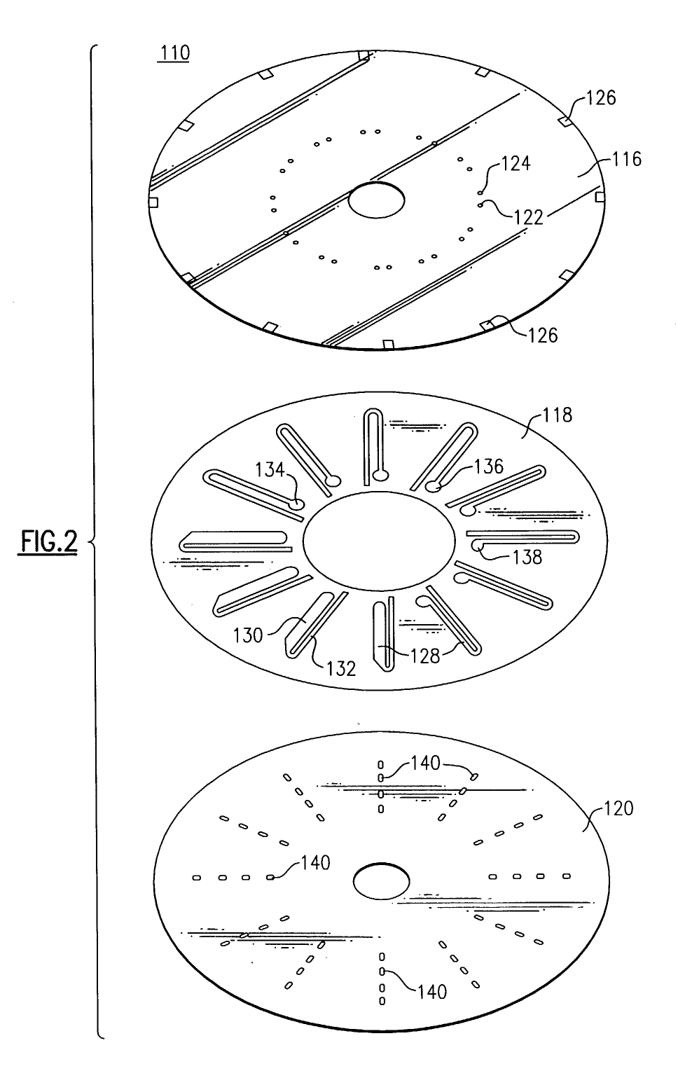

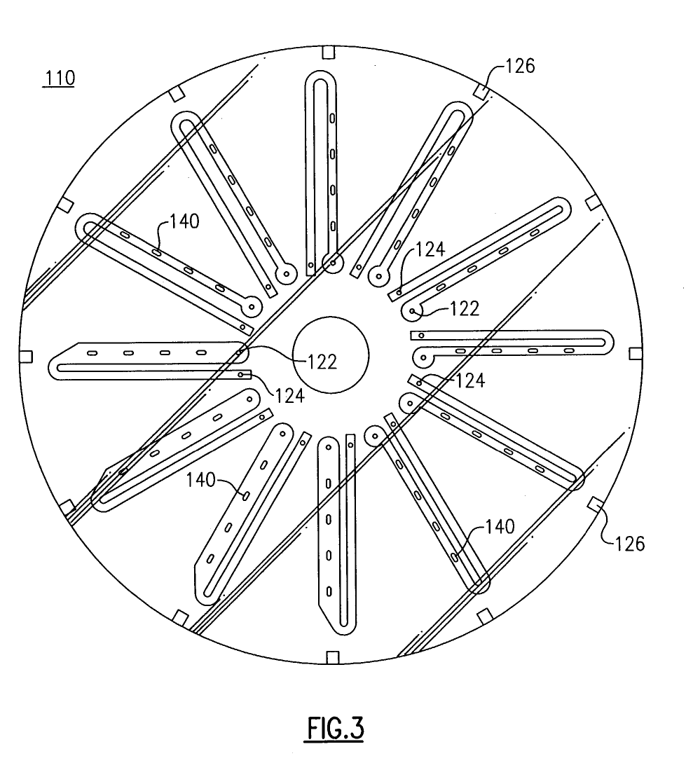

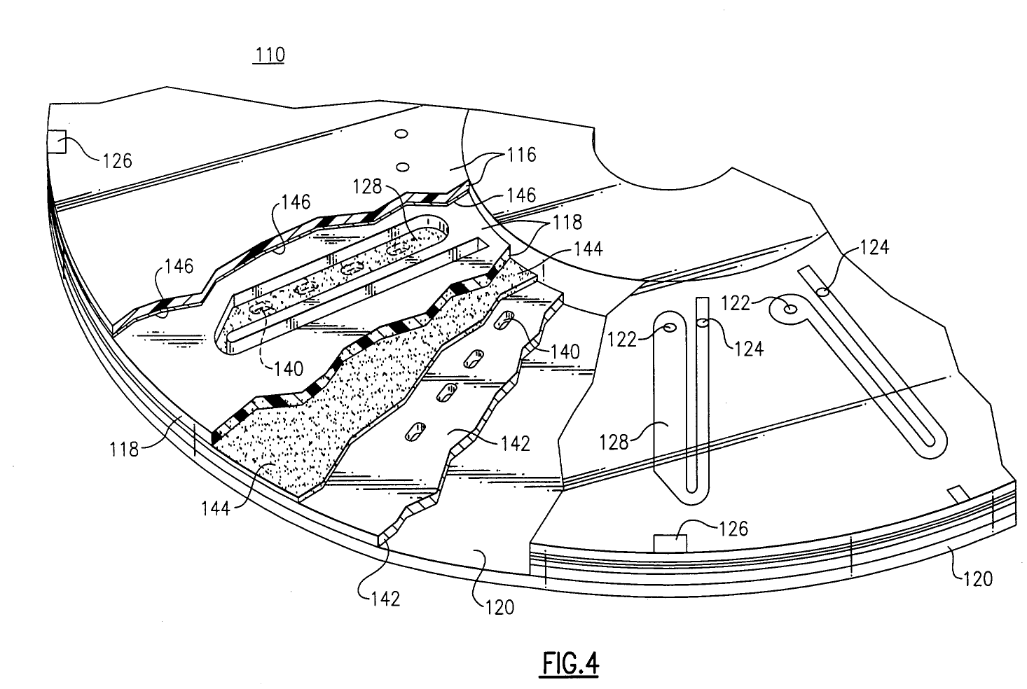

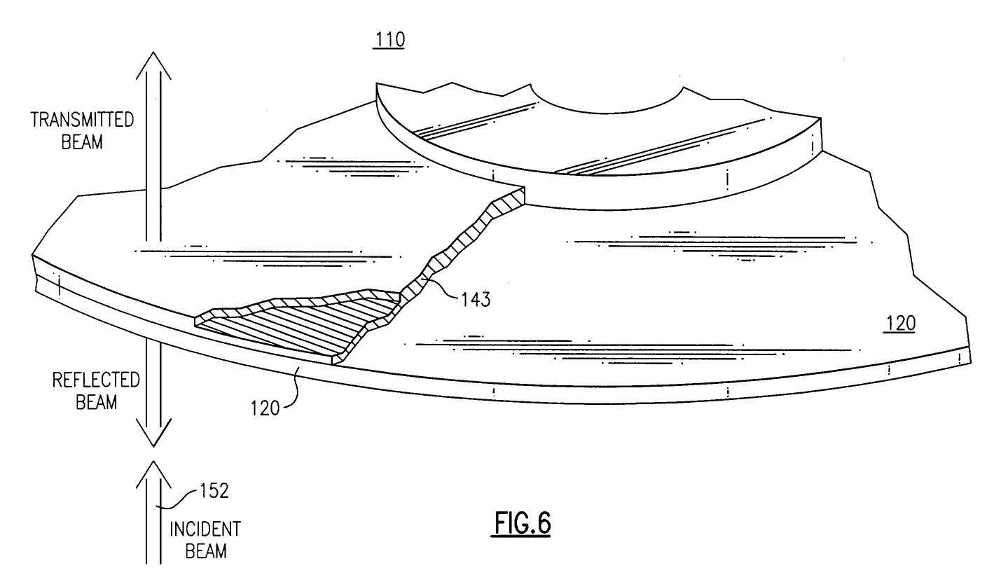

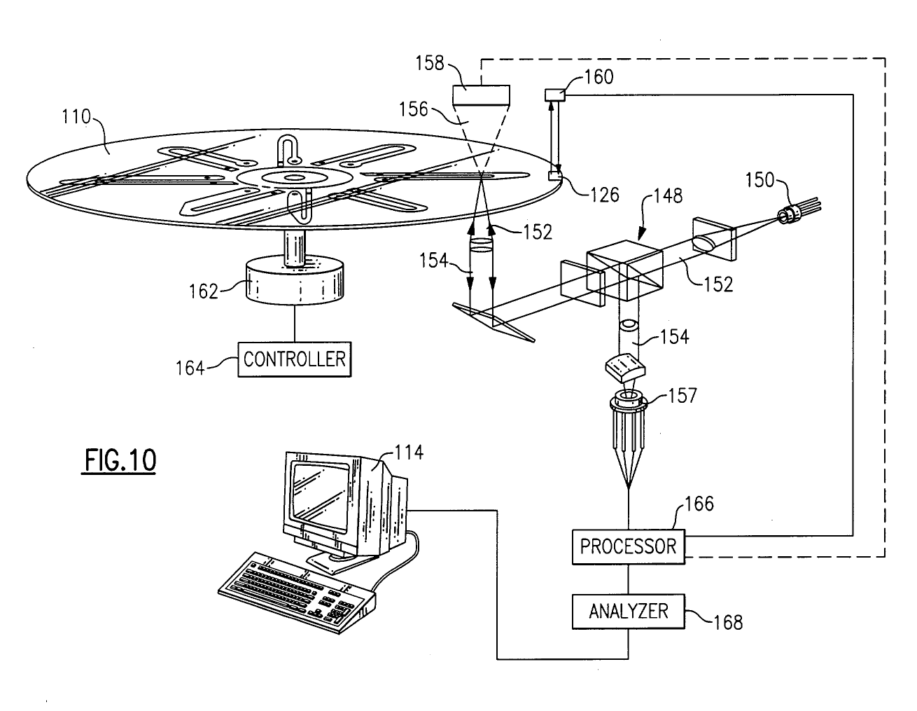

FIG. 2 is an exploded perspective view of the principal structural elements of one embodiment of the optical bio-disc 110. FIG. 2 is an example of a reflective zone optical bio-disc 110 (hereinafter “reflective disc”) that may be used in the present invention. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and is preferably coated with a reflective surface 146 (FIG. 4) on the bottom thereof as viewed from the perspective of FIG. 2. In the preferred embodiment, trigger marks or markings 126 are included on the surface of the reflective layer 142 (FIG. 4). Trigger markings 126 may include a clear window in all three layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the present invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

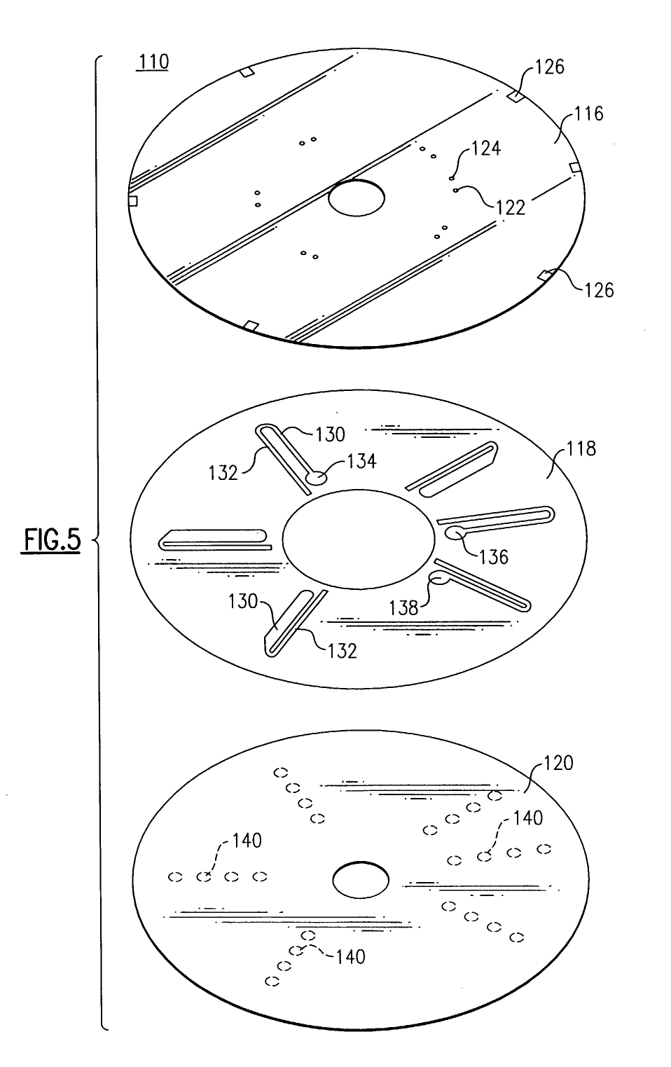

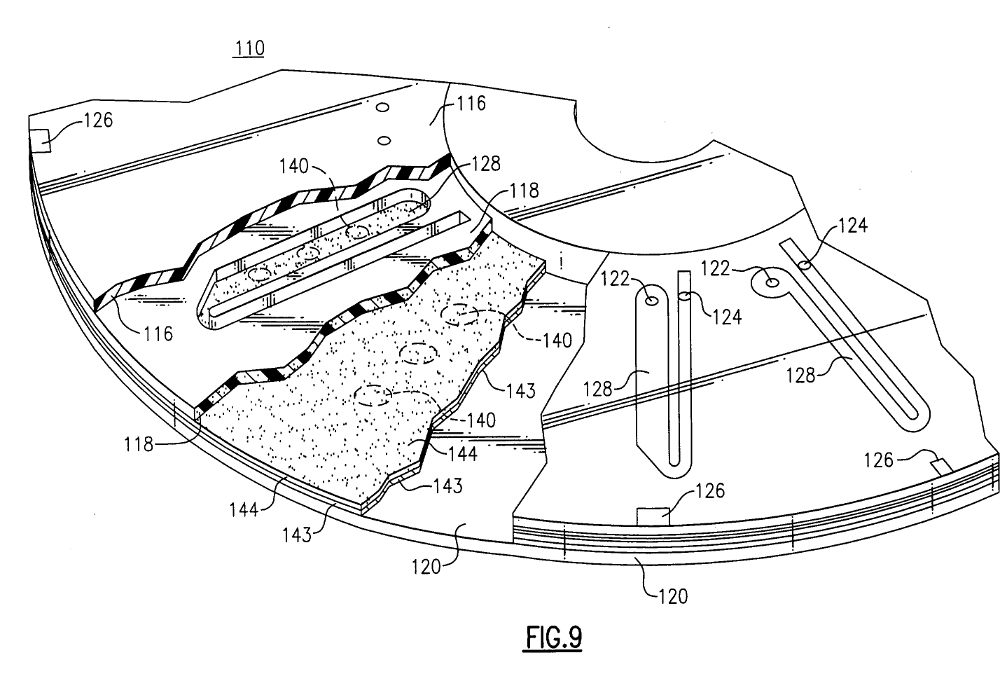

Referring now to FIG. 5, there is shown an exploded perspective view of the principal structural elements of a transmissive type of optical bio-disc 110 according to the present invention. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

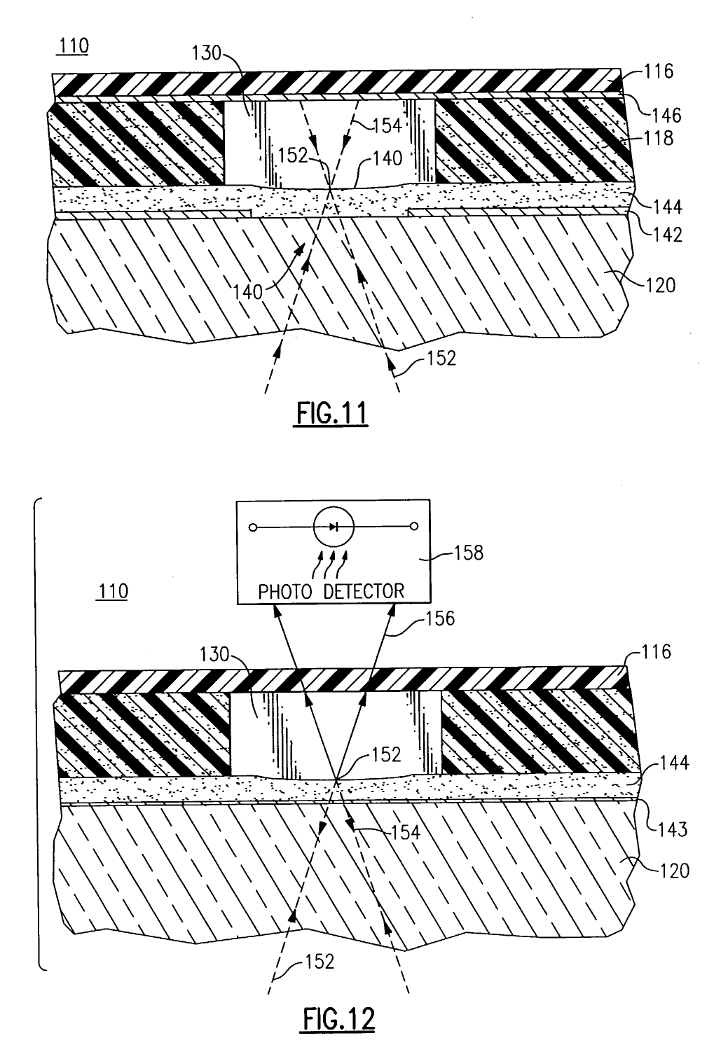

The third element illustrated in FIG. 5 is the substrate 120 which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158, FIGS. 10 and 12, while some of the light is reflected or returned back along the incident path. As indicated below, Table 2 presents the reflective and transmissive characteristics of a gold film relative to the thickness of the film. The gold film layer is fully reflective at a thickness greater than 800 Å. While the threshold density for transmission of light through the gold film is approximately 400 Å.

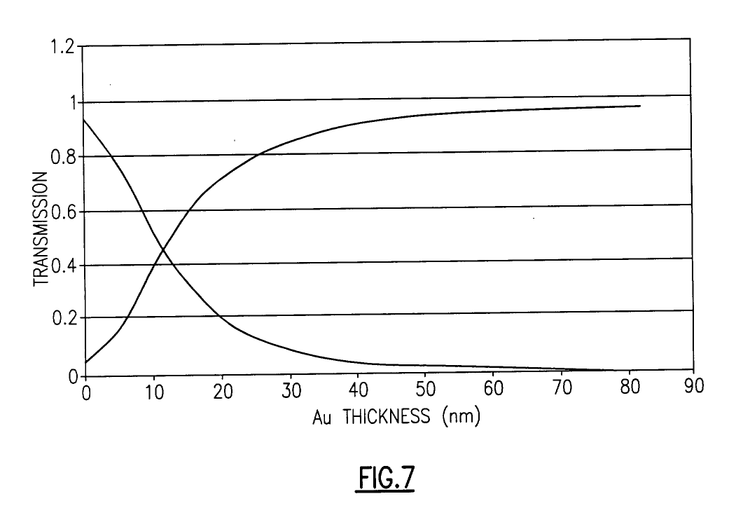

In addition to Table 2, FIG. 7 provides a graphical representation of the inverse relationship of the reflective and transmissive nature of the thin semi-reflective layer 143 based upon the thickness of the gold. Reflective and transmissive values used in the graph illustrated in FIG. 7 are absolute values.

TABLE 2
Au film Reflection and Transmission (Absolute Values)

| Thickness | | | |
|---|---|---|---|
| (Angstroms) | Thickness (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

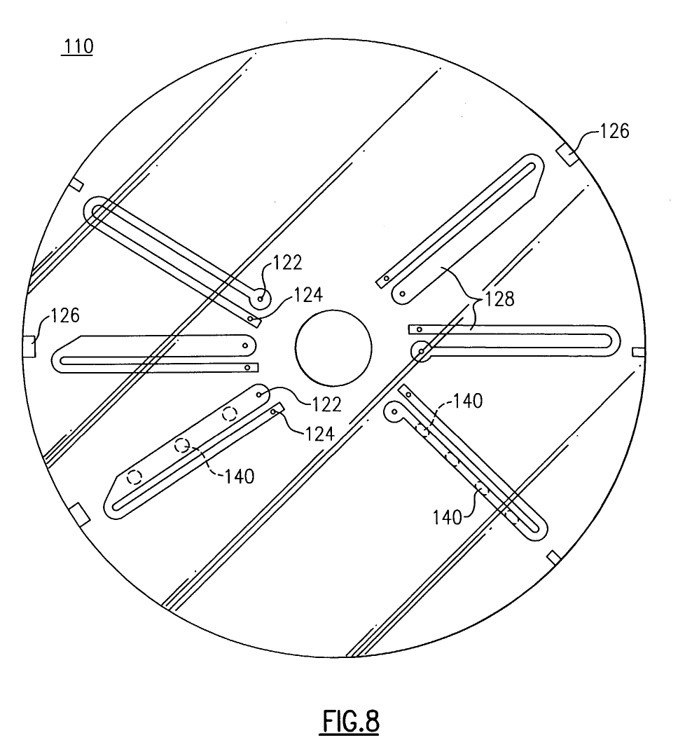

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the present invention. The disc 110 is illustrated with a portion of the various layers thereof cut away to show a partial sectional view of each principal layer, substrate, coating, or membrane. FIG. 9 illustrates a transmissive disc format with the clear cap portion 116, the thin semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 16, FIG. 10. FIG. 9 also shows, the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by a top detector 158, and is also analyzed for the presence of signal elements. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated the transmissive optical bio-disc.

As shown in FIG. 11, there is presented a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

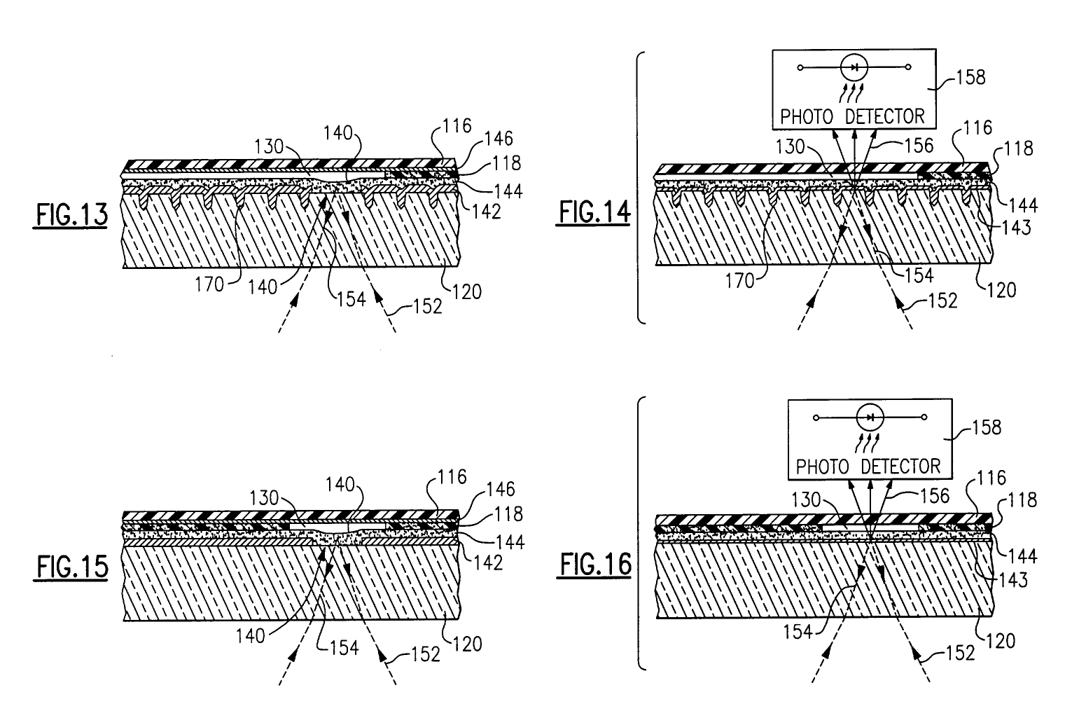

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass there through substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

Counting Methods and Related Software

By way of illustrative background, a number of methods and related algorithms for white blood cell counting using optical disc data are herein discussed in further detail. These methods and related algorithms are not limited to counting white blood cells, but may be readily applied to conducting counts of any type of particulate matter including, but not limited to, red blood cells, white blood cells, beads, and any other objects, both biological and non-biological, that produce similar optical signatures that can be detected by an optical reader.

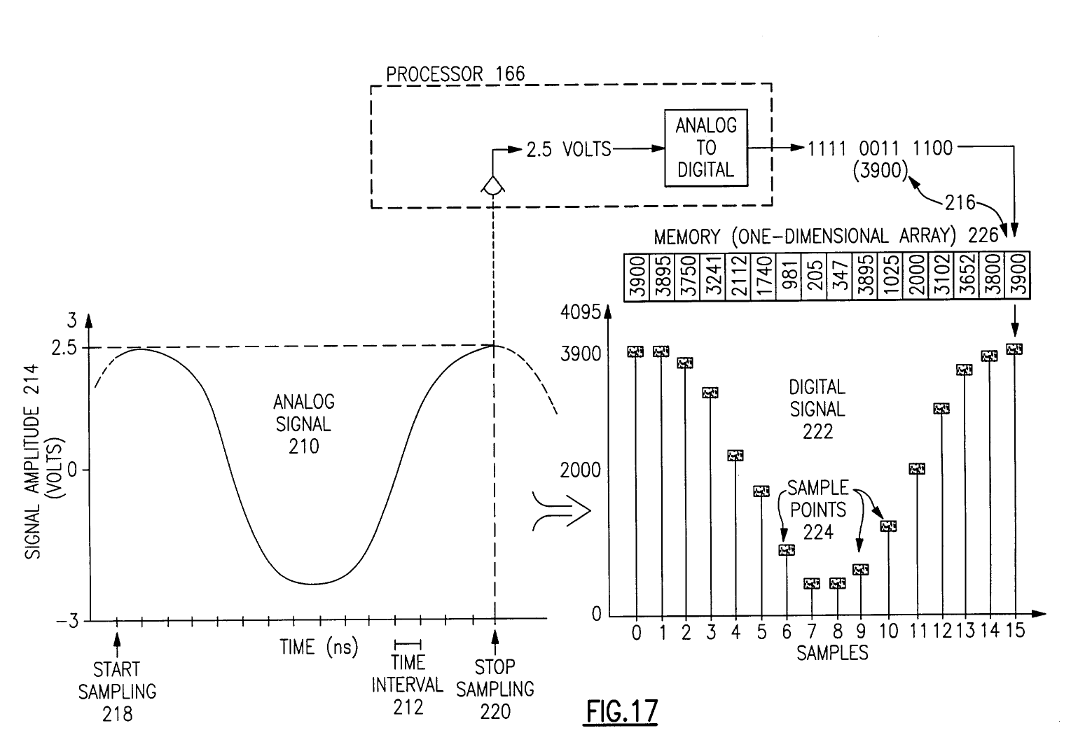

For the purposes of illustration, the following description of the methods and algorithms related to the present invention as described with reference to FIGS. 17-21, are directed to cell counting. With some modifications, these methods and algorithms can be applied to counting other types of objects similar in size to cells. The data evaluation aspects of the cell counting methods and algorithms are described generally herein to provide related background for the methods and apparatus of the present invention. Methods and algorithms for capturing and processing investigational data from the optical bio-disc has general broad applicability and has been disclosed in further detail in commonly assigned U.S. Provisional Application Ser. No. 60/291,233 entitled “Variable Sampling Control For Rendering Pixelation of Analysis Results In Optical Bio-Disc Assembly And Apparatus Relating Thereto” filed May 16, 2001 which is herein incorporated by reference and the above incorporated U.S. Provisional Application Ser. No. 60/404,921 entitled “Methods For Differential Cell Counts Including Related Apparatus And Software For Performing Same”. In the following discussion, the basic scheme of the methods and algorithms with a brief explanation is presented. As illustrated in FIG. 10, information concerning attributes of the biological test sample is retrieved from the optical bio-disc 110 in the form of a beam of electromagnetic radiation that has been modified or modulated by interaction with the test sample. In the case of the reflective optical bio-disc discussed in conjunction with FIGS. 2, 3, 4, 11, 13, and 15, the return beam 154 carries the information about the biological sample. As discussed above, such information about the biological sample is contained in the return beam essentially only when the incident beam is within the flow channel 130 or target zones 140 and thus in contact with the sample. In the reflective embodiment of the bio-disc 110, the return beam 154 may also carry information encoded in or on the reflective layer 142 or otherwise encoded in the wobble grooves 170 illustrated in FIGS. 13 and 14. As would be apparent to one of skill in the art, pre-recorded information is contained in the return beam 154 of the reflective disc with target zones, only when the corresponding incident beam is in contact with the reflective layer 142. Such information is not contained in the return beam 154 when the incident beam 152 is in an area where the information bearing reflective layer 142 has been removed or is otherwise absent. In the case of the transmissive optical bio-disc discussed in conjunction with FIGS. 5, 6, 8, 9, 12, 14, and 16, the transmitted beam 156 carries the information about the biological sample.

With continuing reference to FIG. 10, the information about the biological test sample, whether it is obtained from the return beam 154 of the reflective disc or the transmitted beam 156 of the transmissive disc, is directed to a processor 166 for signal processing. This processing involves transformation of the analog signal detected by the bottom detector 157 (reflective disc) or the top detector 158 (transmissive disc) to a discrete digital form.

Referring next to FIG. 17, the signal transformation involves sampling the analog signal 210 at fixed time intervals 212, and encoding the corresponding instantaneous analog amplitude 214 of the signal as a discrete binary integer 216. Sampling is started at some start time 218 and stopped at some end time 220. The two common values associated with any analog-to-digital conversion process are sampling frequency and bit depth. The sampling frequency, also called the sampling rate, is the number of samples taken per unit time. A higher sampling frequency yields a smaller time interval 212 between consecutive samples, which results in a higher fidelity of the digital signal 222 compared to the original analog signal 210. Bit depth is the number of bits used in each sample point to encode the sampled amplitude 214 of the analog signal 210. The greater the bit depth, the better the binary integer 216 will approximate the original analog amplitude 214. In the present embodiment, the sampling rate is 8 MHz with a bit depth of 12 bits per sample, allowing an integer sample range of 0 to 4095 (0 to (2ⁿ−1), where n is the bit depth. This combination may change to accommodate the particular accuracy necessary in other embodiments. By way of example and not limitation, it may be desirable to increase sampling frequency in embodiments involving methods for counting beads, which are generally smaller than cells. The sampled data is then sent to processor 166 for analog-to-digital transformation.

During the analog-to-digital transformation, each consecutive sample point 224 along the laser path is stored consecutively on disc or in memory as a one-dimensional array 226. Each consecutive track contributes an independent one-dimensional array, which yields a two-dimensional array 228 (FIG. 20A) that is analogous to an image.

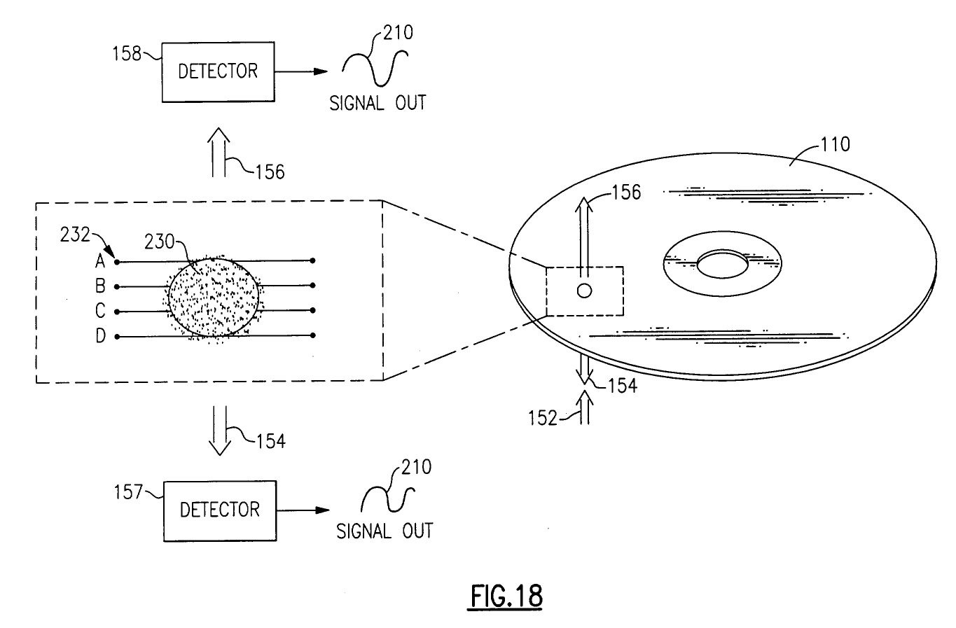

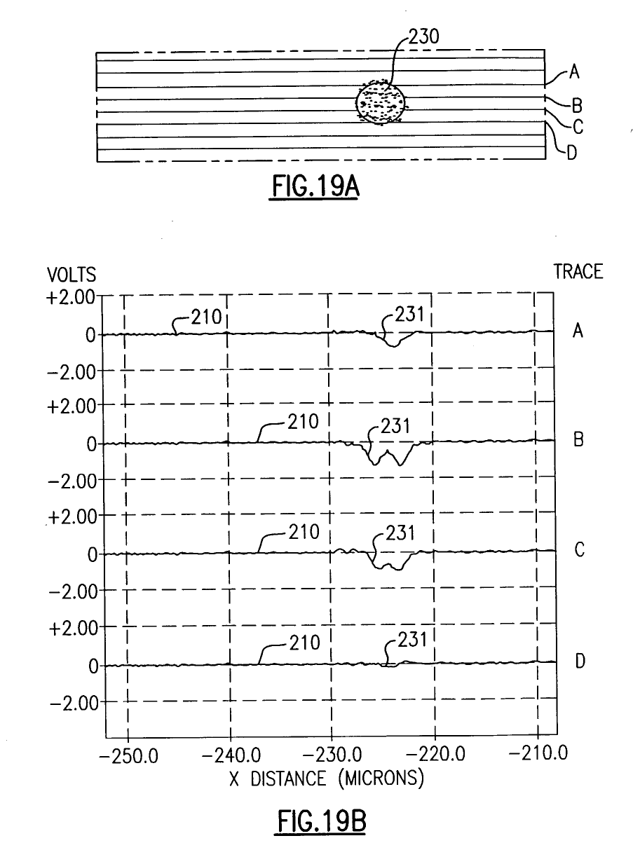

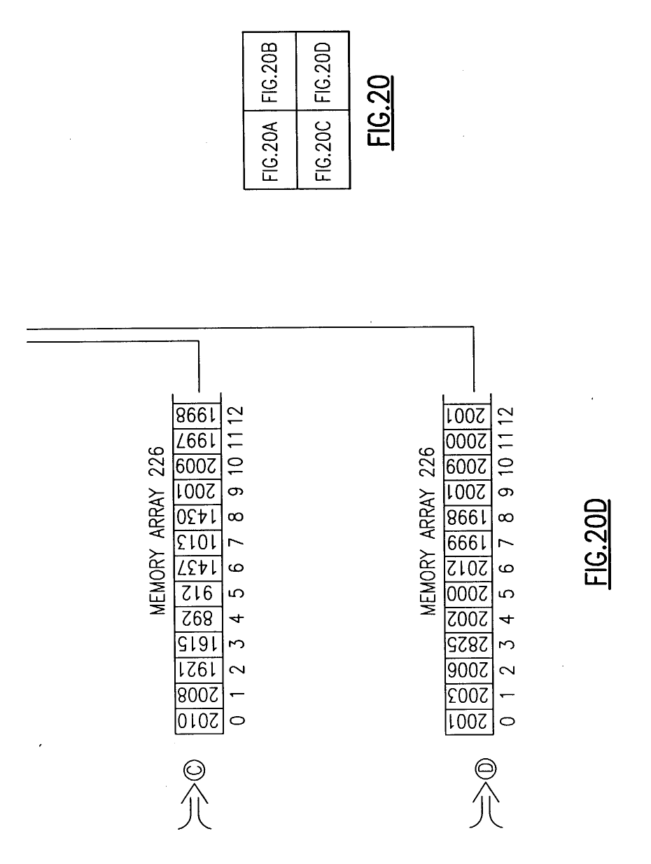

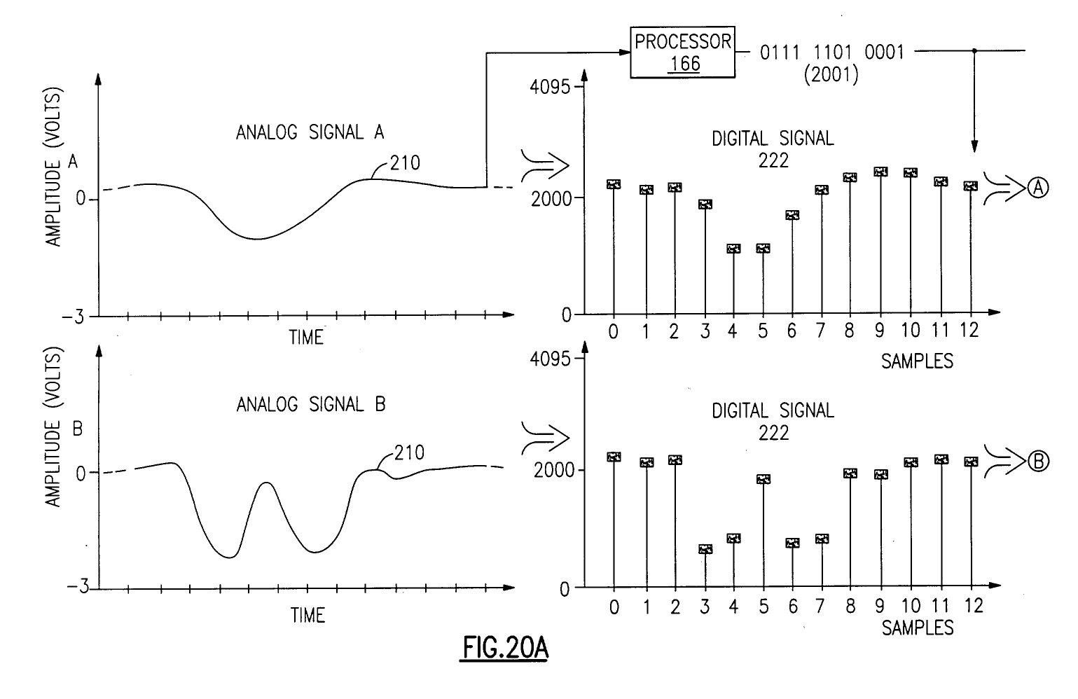

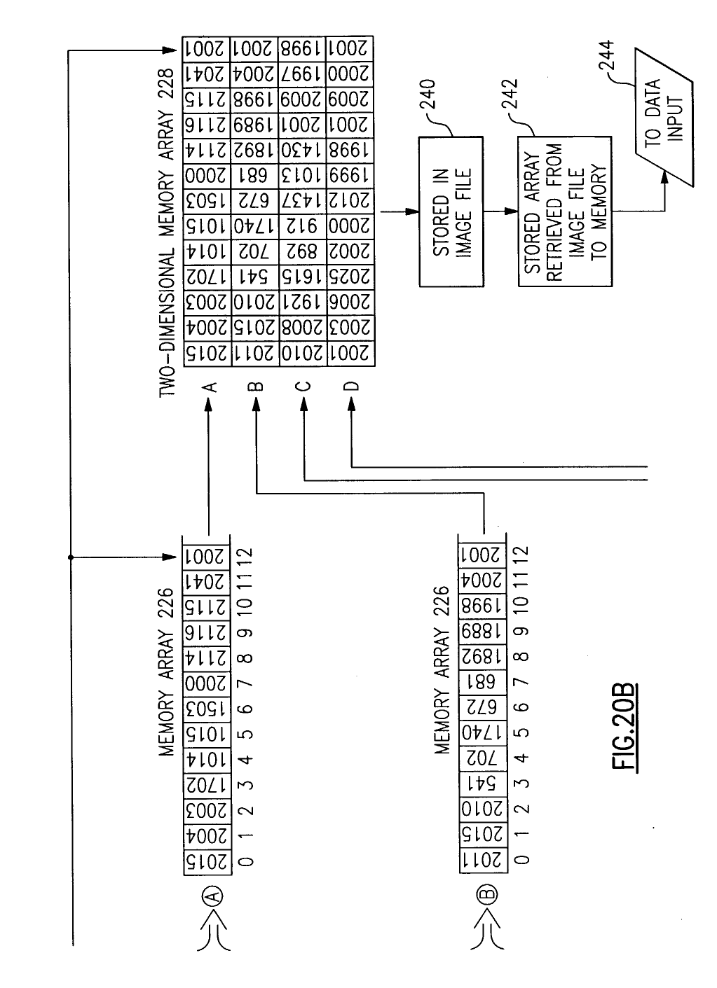

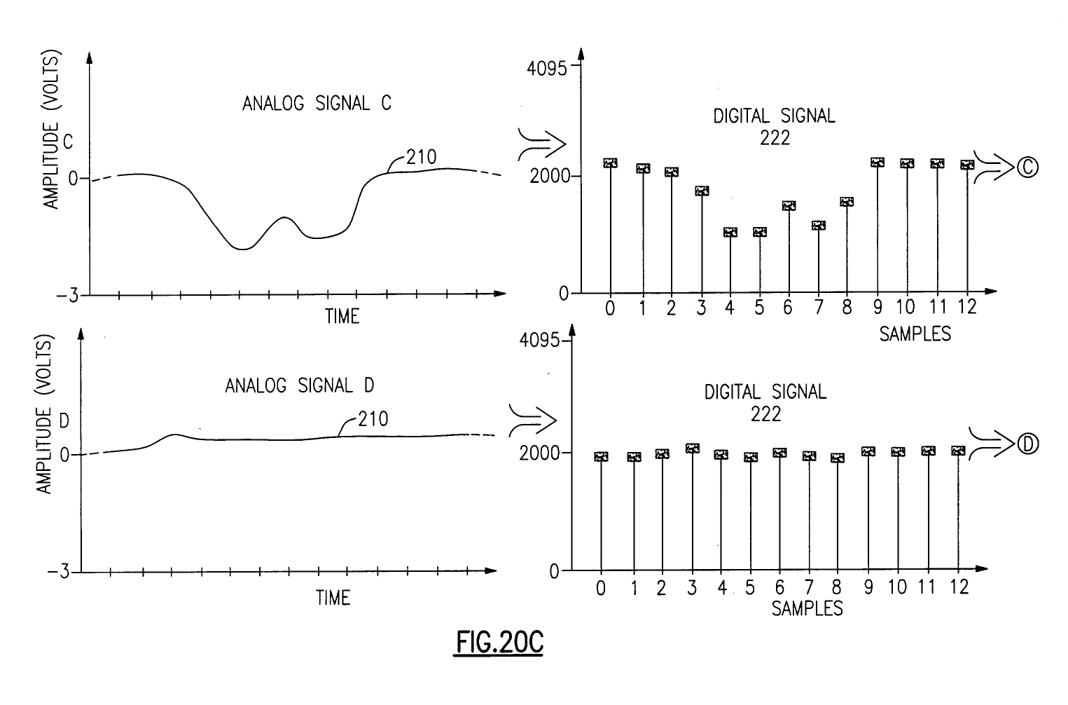

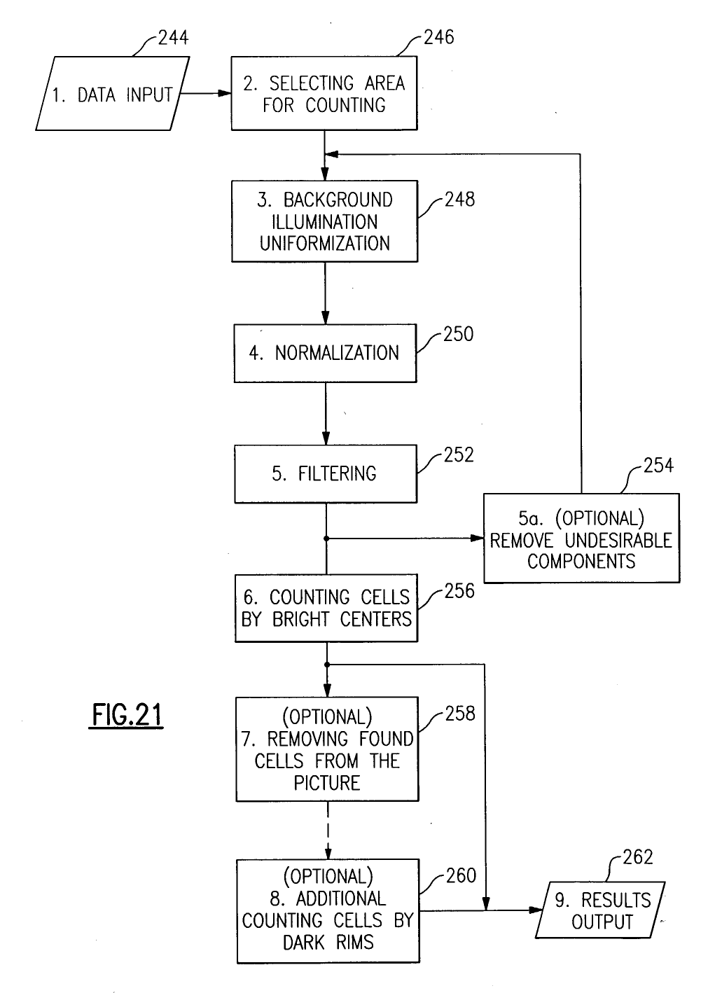

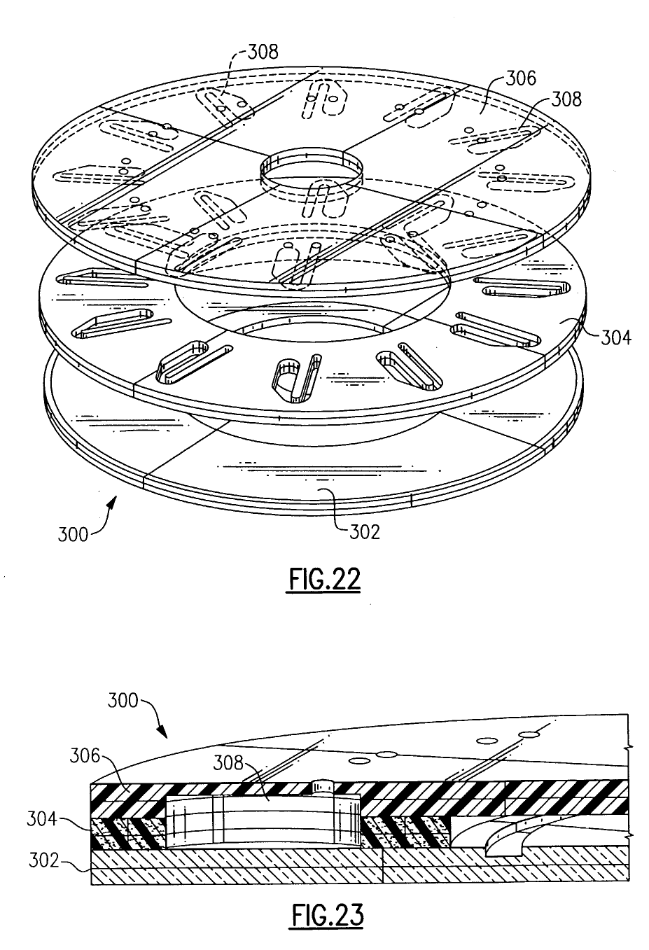

FIG. 18 is a perspective view of an optical bio-disc 110 of the present invention with an enlarged detailed perspective view of the section indicated showing a captured white blood cell 230 positioned relative to the tracks 232 of the optical bio-disc. The white blood cell 230 is used herein for illustrative purposes only. As indicated above, other objects or investigational features such as beads or agglutinated matter may be utilized herewith. As shown, the interaction of incident beam 152 with white blood cell 230 yields a signal-containing beam, either in the form of a return beam 154 of the reflective disc or a transmitted beam 156 of the transmissive disc, which is detected by either of detectors 157 or 158.

FIG. 19A is another graphical representation of the white blood cell 230 positioned relative to the tracks 232 of the optical bio-disc 110 shown in FIG. 18. As shown in FIGS. 18 and 19A, the white blood cell 230 covers approximately four tracks A, B, C, and D. FIG. 19B shows a series of signature traces derived from the white blood cell 210 of FIGS. 19 and 19A. As indicated in FIG. 19B, the detection system provides four analogue signals A, B, C, and D corresponding to tracks A, B, C, and D. As further shown in FIG. 19B, each of the analogue signals A, B, C, and D carries specific information about the white blood cell 230. Thus as illustrated, a scan over a white blood cell 230 yields distinct perturbations of the incident beam that can be detected and processed. The analog signature traces (signals) 210 are then directed to processor 166 for transformation to an analogous digital signal 222 as shown in FIGS. 20A and 20C as discussed in further detail below.

FIG. 20 is a graphical representation illustrating the relationship between FIGS. 20A, 20B, 20C, and 20D. FIGS. 20A, 20B, 20C, and 20D are pictorial graphical representations of transformation of the signature traces from FIG. 19B into digital signals 222 that are stored as one-dimensional arrays 226 and combined into a two-dimensional array 228 for data input 244.

With particular reference now to FIG. 20A, there is shown sampled analog signals 210 from tracks A and B of the optical bio-disc shown in FIGS. 18 and 19A. Processor 166 then encodes the corresponding instantaneous analog amplitude 214 of the analog signal 210 as a discrete binary integer 216 (see FIG. 17). The resulting series of data points is the digital signal 222 that is analogous to the sampled analog signal 210.

Referring next to FIG. 20B, digital signal 222 from tracks A and B (FIG. 20A) is stored as an independent one-dimensional memory array 226. Each consecutive track contributes a corresponding one-dimensional array, which when combined with the previous one-dimensional arrays, yields a two-dimensional array 228 that is analogous to an image. The digital data is then stored in memory or on disc as a two-dimensional array 228 of sample points 224 (FIG. 17) that represent the relative intensity of the return beam 154 or transmitted beam 156 (FIG. 18) at a particular point in the sample area. The two-dimensional array is then stored in memory or on disc in the form of a raw file or image file 240 as represented in FIG. 20B. The data stored in the image file 240 is then retrieved 242 to memory and used as data input 244 to analyzer 168 shown in FIG. 10.

FIG. 20C shows sampled analog signals 210 from tracks C and D of the optical bio-disc shown in FIGS. 18 and 19A. Processor 166 then encodes the corresponding instantaneous analog amplitude 214 of the analog signal 210 as a discrete binary integer 216 (FIG. 17). The resulting series of data points is the digital signal 222 that is analogous to the sampled analog signal 210.

Referring now to FIG. 20D, digital signal 222 from tracks C and D is stored as an independent one-dimensional memory array 226. Each consecutive track contributes a corresponding one-dimensional array, which when combined with the previous one-dimensional arrays, yields a two-dimensional array 228 that is analogous to an image. As above, the digital data is then stored in memory or on disc as a two-dimensional array 228 of sample points 224 (FIG. 17) that represent the relative intensity of the return beam 154 or transmitted beam 156 (FIG. 18) at a particular point in the sample area. The two-dimensional array is then stored in memory or on disc in the form of a raw file or image file 240 as shown in FIG. 20B. As indicated above, the data stored in the image file 240 is then retrieved 242 to memory and used as data input 244 to analyzer 168 FIG. 10.

The computational and processing algorithms of the present invention are stored in analyzer 168 (FIG. 10) and applied to the input data 244 to produce useful output results 262 (FIG. 21) that may be displayed on the display monitor 114 (FIG. 10).

With reference now to FIG. 21 there is shown a logic flow chart of the principal steps for data evaluation according to the processing methods and computational algorithms related to the present invention. A first principal step of the present processing method involves receipt of the input data 244. As described above, data evaluation starts with an array of integers in the range of 0 to 4096.

The next principle step 246 is selecting an area of the disc for counting. Once this area is defined, an objective then becomes making an actual count of all white blood cells contained in the defined area. The implementation of step 246 depends on the configuration of the disc and user's options. By way of example and not limitation, embodiments of the invention using discs with windows such as the target zones 140 shown in FIGS. 2 and 5, the software recognizes the windows and crops a section thereof for analysis and counting. In one preferred embodiment, such as that illustrated in FIG. 2, the target zones or windows have the shape of 1×2 mm rectangles with a semicircular section on each end thereof. In this embodiment, the software crops a standard rectangle of 1×2 mm area inside a respective window. In an aspect of this embodiment, the reader may take several consecutive sample values to compare the number of cells in several different windows.

In embodiments of the invention using a transmissive disc without windows, as shown in FIGS. 5, 6, 8, and 9, step 246 may be performed in one of two different manners. The position of the standard rectangle is chosen either by positioning its center relative to a point with fixed coordinates, or by finding reference mark which may be a spot of dark dye. In the case where a reference mark is employed, a dye with a desired contrast is deposited in a specific position on the disc with respect to two clusters of cells. The optical disc reader is then directed to skip to the center of one of the clusters of cells and the standard rectangle is then centered around the selected cluster.

As for the user options mentioned above in regard to step 246, the user may specify a desired sample area shape for cell counting, such as a rectangular area, by direct interaction with mouse selection or otherwise. In the present embodiment of the software, this involves using the mouse to click and drag a rectangle over the desired portion of the optical bio-disc-derived image that is displayed on a monitor 114. Regardless of the evaluation area selection method, a respective rectangular area is evaluated for counting in the next step 248.

The third principal step in FIG. 21 is step 248, which is directed to background illumination uniformization. This process corrects possible background uniformity fluctuations caused in some hardware configurations. Background illumination uniformization offsets the intensity level of each sample point such that the overall background, or the portion of the image that is not cells, approaches a plane with an arbitrary background value V_{background}. While V_{background} may be decided in many ways, such as taking the average value over the standard rectangular sample area, in the present embodiment, the value is set to 2000. The value V at each point P of the selected rectangular sample area is replaced with the number (V_{background}+(V−average value over the neighborhood of P)) and truncated, if necessary, to fit the actual possible range of values, which is 0 to 4095 in a preferred embodiment of the present invention. The dimensions of the neighborhood are chosen to be sufficiently larger than the size of a cell and sufficiently smaller than the size of the standard rectangle.

The next step in the flow chart of FIG. 21 is a normalization step 250. In conducting normalization step 250, a linear transform is performed with the data in the standard rectangular sample area so that the average becomes 2000 with a standard deviation of 600. If necessary, the values are truncated to fit the range 0 to 4096. This step 250, as well as the background illumination uniformization step 248, makes the software less sensitive to hardware modifications and tuning. By way of example and not limitation, the signal gain in the detection circuitry, such as top detector 158 (FIG. 18), may change without significantly affecting the resultant cell counts.

As shown in FIG. 21, a filtering step 252 is next performed. For each point P in the standard rectangle, the number of points in the neighborhood of P, with dimensions smaller than indicated in step 248, with values sufficiently distinct from V_{background} is calculated. The points calculated should approximate the size of a cell in the image. If this number is large enough, the value at P remains as it was; otherwise it is assigned to V_{background}. This filtering operation is performed to remove noise, and in the optimal case only cells remain in the image while the background is uniformly equal V_{background}.

An optional step 254 directed to removing bad components may be performed as indicated in FIG. 21. Defects such as scratches, bubbles, dirt, and other similar irregularities may pass through filtering step 252. These defects may cause cell counting errors either directly or by affecting the overall distribution in the images histogram. Typically, these defects are sufficiently larger in size than cells and can be removed in step 254 as follows. First a binary image with the same dimensions as the selected region is formed. A in the binary image is defined as white, if the value at the corresponding point of the original image is equal to V_{background}, and black otherwise. Next, connected components of black points are extracted. Then subsequent erosion and expansion are applied to regularize the view of components. And finally, components that are larger than a defined threshold are removed. In one embodiment of this optional step, the component is removed from the original image by assigning the corresponding sample points in the original image with the value V_{background}. The threshold that determines which components constitute countable objects and which are to be removed is a user-defined value. This threshold may also vary depending on the investigational feature being counted i.e. white blood cells, red blood cells, or other biological matter. After optional step 254, steps 248, 250, and 252 are preferably repeated.

The next principal processing step shown in FIG. 21 is step 256, which is directed to counting cells by bright centers. The counting step 256 consists of several substeps. The first of these substeps includes performing a convolution. In this convolution substep, an auxiliary array referred to as a convolved picture is formed. The value of the convolved picture at point P is the result of integration of a picture after filtering in the circular neighborhood of P. More precisely, for one specific embodiment, the function that is integrated, is the function that equals v-2000 when v is greater than 2000 and 0 when v is less than or equal to 2000. The next substep performed in counting step 256 is finding the local maxima of the convolved picture in the neighborhood of a radius about the size of a cell. Next, duplicate local maxima with the same value in a closed neighborhood of each other are avoided. In the last substep in counting step 256, the remaining local maxima are declared to mark cells.

In some hardware configurations, some cells may appear without bright centers. In these instances, only a dark rim is visible and the following two optional steps 258 and 260 are useful.

Step 258 is directed to removing found cells from the picture. In step 258, the circular region around the center of each found cell is filled with the value 2000 so that the cells with both bright centers and dark rims would not be found twice.

Step 260 is directed to counting additional cells by dark rims. Two transforms are made with the image after step 258. In the first substep of this routine, substep (a), the value v at each point is replaced with (2000-v) and if the result is negative it is replaced with zero. In substep (b), the resulting picture is then convolved with a ring of inner radius R1 and outer radius R2. R1 and R2 are, respectively, the minimal and the maximal expected radius of a cell, the ring being shifted, subsequently, in substep (d) to the left, right, up and down. In substep (c), the results of four shifts are summed. After this transform, the image of a dark rim cell looks like a four petal flower. Finally in substep (d), maxima of the function obtained in substep (c) are found in a manner to that employed in counting step 256. They are declared to mark cells omitted in step 256.

After counting step 256, or after counting step 260 when optionally employed, the last principal step illustrated in FIG. 21 is a results output step 262. The number of cells found in the standard rectangle is displayed on the monitor 114 shown in FIGS. 1 and 5, and each cell identified is marked with a cross on the displayed optical bio-disc-derived image.

Additional computer science methodologies and apparatus directed to extracting and visualizing data from bio-discs and/or optical analysis discs are discussed in commonly assigned U.S. patent application Ser. No. 10/______ entitled “Method and Apparatus for Visualizing Data” filed Jan. 13, 2003 and U.S. patent application Ser. No. 10/______ entitled “Methods and Apparatus for Extracting Data From an Optical Analysis Disc” filed on Jan. 14, 2003 both of which have been herein incorporated by reference.

Processes for Manufacturing Discs with Molded Micro-fluidic Structures and Discs Made According Thereto

This invention provides a process for manufacturing an optical analysis disc (also referred to as a BioCD, optical bio-disc, or compact bio-disc) with microfluidic structures molded into a cover disc bonded onto lens disc by patterned adhesive as intermediate layer. The invention is also related to the discs made according thereto.

The BioCD is in a Compact Disc format with disc diameter of 120 mm (maybe of other diameters in other versions, such as 80 mm in mini disc). FIG. 22 illustrates the integration structure 300 of disc. The lens disc 302, operating as substrate, is made through industrial CD manufacturing process with fully reflective or partial transmissive metal layer on top. The intermediate layer 304 is patterned adhesive deposited on lens disc. The top layer 306 is the disc with molded microfluidic structures 308 inside.

In particular, the disc structure includes a lens disc component 302 with signaling tracks on disc; patterned adhesive 304 as intermediate layer for disc bonding, and a cover disc 306 with molded microfluidic structures 308.

This BioCD can provide optical quality with molded microfluidic structures and chemistry deposition step can be easily integrated into the manufacturing process.

The intermediate layer 304 is adhesive that can be applied on lens disc 302 with designed patterns. The pattern corresponds to both the capturing chemistry spots as well as molded microfluidic cover disc. Various types of adhesive and related curing methods can be adopted to serve for this purpose. In both FIGS. 22 and 23 the correspondence of patterned adhesive and molded fluidic structures can be appreciated.

The manufacturing procedure for the molded cover disc according to the present invention include the following particular aspects.

Design of desired fluidic structures by computer drawing software, such as AutoCAD, and then making the respective mask made of chrome glass by e-beam patterning or a Scitex® film printed with high-resolution film printer.

A mastering support such as a mastering glass is prepared by processes of cleaning and photoresist coating. Various types of photoresist can be used in this operation, thick photoresist such as AZ5412, AZ4620, SU8-5, SU8-25, SU8-50, SU8-100. Photoresist coating process can be done by dipping, spray coating, and spin coating.

It should be noted that the cleaning process is critical to ensure the cleanness of operation.

Accordingly, a lithography process to transfer design pattern onto mastering glass and develop the photoresist is carried out. The lithography operation may involve the use of the following instruments: a SCS Spin coater, a hotplate, an OAI Mask Aligner, and a fume hood.

The following chemicals can be used. Photo resist selected in a group consisting of AZ5412, AZ4620, SU8-5, SU8-25, SU8-50, SU8-100; a surface adhesion promoter; a developer, e.g. AZ 400K developer and SU8 developer; a thinner, e.g. SU8 thinner; a remover, e.g. SU8 remover; Isopropanol, acetone, methanol; deionized water (0.2 um filtration); sulfuric acid; hydrogen peroxide (30%); nitrogen gas; and dry air.

A substrate is prepared, i.e. either mastering glass (soda lime) or silicon wafer. This substrate is cleaned by immersion in a solution of H₂SO₄- H₂O₂ (7:1- 10:1) and then taken out, rinsed with deionized water and blown dry.

A dehydration baking is performed, with the hotplate setting at 150 degree C. for 15-20 minutes. Then, the substrate is cooled down to room temperature, avoiding a temperature shock to the substrate. Optionally, a surface adhesion promoter onto substrate can be applied.

Thereafter, the substrate can be mounted onto a coater device, i.e. a spin coater, to apply a photoresist composition covering at least ⅔ of surface. Care should be taken when applying photoresist to avoid bubble formation during coating process. Dust should be eliminated also.

A further soft-baking can be performed at the hotplate to remove solvent in photoresist. The baking temperature and duration are based on type and thickness of photoresist used.

On the above substrate, i.e. the mastering glass, the designed mask is applied and a UV exposure is carried out to activate the photoresist composition. The exposure duration is based on required dosage of photoresist versus UV exposure intensity. After pattern is developed, rinsing and blow dry need to be carried out.

Once the above described cycle of lithography operation is completed, the substrate is ready to continue with electroplating or another operation of lithography. The above-mentioned processes can be repeated if multi-layer of fluidic structures is required provided that multi-layer fluidic structures require separate sets of mask design.

Once the mastering glass is ready, a seed layer of metal, e.g. silver or nickel, is deposited by vacuum deposition and then an electroplating process is continued to obtain a plated stamper.

After plating cycle completed, the plated stamper is peeled off from mastering glass and continued with backside polishing process. Backside of stamper may have undesired cavities due to the design pattern which can be removed or filled.

It should be noted that the backside polishing and/or cavity filling improve the quality of molded disc during molding process, extending the life-time of the stamper and thus reducing production costs.

After the polishing process the stamper can be mounted onto injection molding machine for disc manufacturing. Molding can be done with injection molding, compression/injection molding, hot embossing with the stamper made.

According to a modified embodiment, also a MEMS (Micro-Electro-Mechanical Systems) process on silicon wafer could be used to produce a suitable stamper, through dry/wet etching process together with lithography operations. The major difference is the resolution versus operation cost. High precision and resolution can be achieved with MEMS operation on silicon wafer. Concluding Statements All patents, provisional applications, patent applications, and other publications mentioned in this specification are incorporated herein in their entireties by reference.

While this invention has been described in detail with reference to a certain preferred embodiments, it should be appreciated that the present invention is not limited to those precise embodiments. Rather, in view of the present optical bio-disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

1. A process for manufacturing optical analysis discs with microfluidic structures, said process including the steps of:
providing a lens disc;
patterning an intermediate adhesive layer with a pattern corresponding to predetermined microfluidic structures;
providing a cover disc having molded microfluidic structures; and
applying said cover disc onto said intermediate adhesive layer so as to match said molded microfluidic structures to said pattern to bond the cover disc onto the lens disc.

2. The process for manufacturing optical analysis discs according to claim 1 wherein said intermediate adhesive layer is patterned according to capturing chemistry spots.

3. The process for manufacturing optical analysis discs according to claim 1 wherein said intermediate adhesive layer is applied by a transfer adhesive technique.

4. The process for manufacturing optical analysis discs according to claim 1 wherein said intermediate adhesive layer is applied by a printing technique.

5. The process for manufacturing optical analysis discs according to claim 3 wherein said intermediate adhesive layer is made of a thermoglue.

6. The process for manufacturing optical analysis discs according to claim 1 wherein the step of providing a cover disc having molded microfluidic structures includes the steps of:
providing a mastering support;
coating the mastering support with a photoresist composition;
transferring onto said mastering support a pattern design of microfluidic structures;
developing said photoresist composition according to said pattern design;
depositing a metal layer onto the mastering support until a plated stamper is obtained;
peeling of said plated stamper; and
molding said microfluidic structures in a cover disc according said plated stamper.

7. The process for manufacturing optical analysis discs according to claim 6 wherein said mastering support is a mastering glass.

8. The process for manufacturing optical analysis discs according to claim 6 wherein said mastering support is prepared by a process of cleaning.

9. The process for manufacturing optical analysis discs according to claim 6 wherein said photoresist composition is selected from the group consisting of AZ5412, AZ4620, SU8-5, SU8-25, SU8-50, and SU8-100.

10. The process for manufacturing optical analysis discs according to claim 6 wherein said coating of a photoresist composition is carried out by dipping, spray coating, or spin coating.

11. The process for manufacturing optical analysis discs according to claim 6 wherein the steps of coating the mastering support with a photoresist composition; transferring onto said mastering support a pattern design of microfluidic structures; and developing said photoresist composition according to said pattern design are repeated to obtain multi-layer microfluidic structures.

12. The process for manufacturing optical analysis discs according to claim 6 wherein the depositing of a metal layer is carried out by vacuum deposition.

13. An optical analysis disc having microfluidic structures, said disc comprising:
a lens disc;
a patterned adhesive as intermediate layer for disc bonding; and
a cover disc with molded microfluidic structures.

14. The optical analysis disc according to claim 13 wherein the lens disc comprises signaling tracks.

15. The optical analysis disc according to claim 13 wherein said patterned adhesive is patterned according to capturing chemistry spots.

16. The optical analysis disc according to claim 13 wherein said patterned adhesive layer is a thermoglue.

17. The process for manufacturing optical analysis discs according to claim 4 wherein said intermediate adhesive layer is made of a thermoglue.
